(19) United States
(12) Patent Application Publication
(10) Pub. No.: US 2007/0093725 A1
(43) Pub. Date: Apr. 26, 2007

(54) Dual entry collection device for breath analysis

(76) Inventor: David I-Hung Shaw, San Francisco, CA (US)

Correspondence Address: JONES DAY, 222 EAST 41ST ST, NEW YORK, NY, 10017, US

(21) Appl. No.: 11/245,001

(22) Filed: Oct. 6, 2005

(51) Int. Cl. A61B 5/08

(52) U.S. Cl. 600/543

(57) ABSTRACT

A breath analyzing apparatus comprising a collection tube that has at least a first and second breathing entry is provided. The collection tube is hollow such that the breathing entries are in fluid communication with each other. A sensor airway tube is fluidly joined to a central portion of the collection tube by a pin-hole orifice. With this design, a small portion of a breath sample is collected by the sensor airway tube when a user blows into either breathing entry and the majority of the breath sample is expunged out of the opposing breathing entry. The sensor airway tube either houses or is proximate to a sensor that senses the composition of the breath sample. Illumination is used to guide a user on where to blow, when to blow, for how long to blow, as well as to inform the user on the result of the breath analysis.

### FIELD OF THE INVENTION

This invention relates to novel breathalyzers and new methods for breath collection and analysis.

### BACKGROUND OF THE INVENTION

Breath samples of animals, especially human breath samples, contain alveolar air, the gas in the pulmonary alveoli where O₂—CO₂ exchange with pulmonary capillary blood occurs. The content of alveolar air provides information for disease diagnosis and drug monitoring. Thus breath collection and breath analysis is of importance.

A blood alcohol analyzing unit, such as a breathalyzer, is one of the most commonly used devices for breath analysis. Available breathalyzers are classified into two types: the “closed-flow” type and the “open-flow” type, defined by whether a breath sample is retained in a closed chamber during analysis. A closed-flow breathalyzer, as illustrated for example in FIG. 1A, contains an easily recognizable inlet 102 for breath intake. In a closed-flow breathalyzer, the user's mouth or lips, or a straw used by the user typically contact the breathalyzer directly. An open-flow breathalyzer, as illustrated for example in FIG. 1B, usually contains an orifice where users can blow into the unit. No straw or any additional device is needed, and the user's mouth or lips do not contact the breathalyzer.

Closed-flow breathalyzers, such as the one illustrated in FIG. 1A use a mouthpiece or a straw (e.g., 102) to stabilize a breath stream before breath analysis takes place. Therefore, they can accurately measure breath samples. However, the drawback with closed-flow breathalyzers is that they are inconvenient to use. The mouthpiece or straw demands constant cleaning or replacement to ensure sanitation. The inherent length of the straw or mouthpiece and the need for either a disposable straw or a replaceable mouthpiece cover compromises the compactness of a close-flow breathalyzer. Closed-flow breathalyzers are often larger in size and cumbersome to handle, store and operate. Furthermore, because breath samples are retained within the closed-form breathalyzer during measurement, residual air (e.g., ambient air or residual breath from previous users) may not be readily cleared out of such breathalyzers. In worse-case situations, when residual breath samples from previous users are not efficiently purged out of the breathalyzer, breath measurements can be severely compromised in breath analysis of subsequent users.

Open-flow breathalyzers, such as the one illustrated in FIG. 1B, do not use an inlet for breath sample intake, instead breath samples are directly blown through an orifice (e.g., orifice 108) onto a sensor for detection without mouth or lip contact with the breathalyzer. As a result, the breath samples are instantly mixed with ambient air. In the open-flow breathalyzers, the sensors are directly exposed to ambient air, or placed behind a grill that is exposed to ambient air. In the absence of an inlet or any additional stabilizing devices, the breath samples are diluted further before measurement takes place at the sensors. Consequently, even though the open-flow breathalyzers eliminate the sanitary concerns associated with the closed-flow breathalyzers, measurements by an open-flow breathalyzers are usually inaccurate and inconsistent.

In both the closed-flow and open-flow breathalyzer designs, inadequate user instructions are typically provided. Often such user instructions are provided in a manual that is lost or otherwise separated from the breathalyzer. As a result, such breathalyzers are often used in a manner that will not afford accurate results. For example, a user will typically ignore or lose user instructions and blow into the breathalyzer in a manner that is not appropriate for the device thereby causing an in accurate measurement.

Given the above background, more convenient easier to use breath collection methods and apparatuses are needed in the art.

### SUMMARY OF THE INVENTION

One aspect of the invention provides a dual entry, self-purging breathalyzer for accurate breath analysis without any requirement for mouth or lip contact by the user. As detailed below, this aspect of the present invention is advantageous over both closed-flow and open-flow breathalyzers.

Some embodiments of the invention provide breath analyzing methods that utilize an apparatus comprising a collection device. The collection device has a first breathing entry and a second breathing entry. The first breathing entry and the second breathing entry are in fluid communication with each other. The collection device has a center between the first breathing entry and the second breathing entry. As used herein, the term “center” means any point between the two ends of the first breathing entry and the second breathing entry. In one inventive method, a user breathes into either the first or second breathing entry thereby delivering a breath sample. A reading that is indicative of a characteristic of the user is then obtained from the apparatus. In some embodiments, the apparatus further comprises one or more sensors collectively configured to measure a physical feature of the breath sample. The sensors are located between the first breathing entry and the second breathing entry of the collection device. In such embodiments, the method further comprises measuring, between the first breathing entry and the second breathing entry of the collection device, a physical feature of the breath sample. In some embodiments, the apparatus further comprises an airway tube in fluid communication with the collection device. Furthermore, in such embodiments, the one or more sensors are located in the airway tube. In some embodiments, the characteristic of the user that is reported by the breathalyzer is user blood alcohol level.

In some embodiments, the collection device is a tube and the first breathing entry is a first opening of the tube while the second breathing entry is a second opening of the tube. In some embodiments, a user blows into the breathalyzer in response to a first visible signal. This first visible signal can be, for example, a first type of illumination (e.g., blue light) that shines in the vicinity of the first and/or second breathing entry. In some embodiments, a second visible signal, such as a second type of illumination (e.g., a red light or a green light) shines in the vicinity of the first breathing entry and the second breathing entry once data acquisition is complete.

A second aspect of the present invention provides a breath analyzing apparatus that includes a collection device. This collection device comprises a first and second breathing entry that are in fluid communication with each other. The collection device has a center between the first and second breathing entry. One or more sensors are collectively configured to measure a physical feature of a breath sample. To do this, the one or more sensors are positioned so that they are in fluid communication with a region of the collection device that is between the first and second breathing entry of the collection device.

In some embodiments in accordance with the second aspect of the invention, the collection device is a tube, the first breathing entry is a first opening of the tube, and the second breathing entry is a second opening of the tube. In some embodiments, the apparatus further comprises a sensor airway tube that is in fluid communication with the collection device. In such embodiments, this sensor airway tube houses the one or more sensors. In some embodiments, the sensor airway tube comprises a first portion having a first inner diameter and a second portion having a second inner diameter. In such embodiments, the first portion of the sensor airway tube is attached to the collection device. In some embodiments, the first inner diameter is smaller than the second inner diameter.

Some embodiments of the present invention have the additional feature of providing one or more illumination sources. The one or more illumination sources can serve any combination of several different purposes. For example, they can be used to inform a user where to blow, when to blow, how long to blow, and the status or the results of the breath analysis. The one or more illumination sources can also provide quick and easy to understand indication of test results (e.g., indicating a pass or fail by a simple color change or by the use of a specific color to indicate a particular result). Such functionality provides a quick and easy visual prompt and conveys status and test results in dark or noisy environments. Thus, a user can obtain results using the inventive apparatus even in situations where the user is either visually or orally impaired. The optional illumination features of the present invention are particularly useful since breathalyzers are often used in loud, noisy bars, parties or public social environments and events. Advantageously, the illumination sources can guide a user into breathing into the device in the appropriate manner even when the surrounding environment is noisy or dim.

In some embodiments in accordance with the second aspect of the invention, the collection device further comprises a first illumination source connected to the first breathing entry as well as a second illumination source connected to the second breathing entry. In such embodiments, a control mechanism is in electrical communication with the first and second illumination sources. This control mechanism is configured to cause the first and second illumination sources to emit a first visible signal (e.g., a blue light) indicating a time for a user to commence breathing into the first or second breathing entry. The control mechanism is also configured to cause the first and second illumination sources to emit a second visible signal (e.g., a green light or a red light) indicating a characteristic of the user after the user has provided the breath sample.

In some embodiments, the control mechanism is configured to continue emitting a first visible signal during a breath measurement. In some embodiments, the control mechanism is configured to emit a signal indicating insufficient breath input. This signal can be an audible alarm, or a message on a display housed by the apparatus.

In some embodiments, the breath analyzing apparatus is a handheld device such as a cell phone or a personal digital assistant (PDA). In some embodiments, the apparatus is a handheld device having a casing and the collection device is housed by the casing in a manner such that the first breathing entry forms a first entry in the casing and the second breathing entry forms a second entry in the casing.

The methods and apparatus of the present invention thus provide superior sanitation relative to closed-flow breathalyzers because, unlike closed-flow breathalyzers, the methods and apparatus of the present invention do not require user mouth contact. Consequently, the methods and apparatus of the present invention do not require regular cleaning or part replacement. Advantageously, in some embodiments, the breathalyzers of the present invention have a symmetrical design that affords ambidextrous use. Also, advantageously, the dual entry design of breath collection devices in accordance with the present invention are self-purging. This means that any lingering breath sample from a previous use is cleared out of the device prior to a subsequent use to ensure accurate measurement. In embodiments where the dual entry breathalyzer of the present invention is self-purging and ambidextrous, the breathalyzer can be passed around quickly without reorientation or shaking to clear out residual breath from previous tests. The design allows for rapid use by multiple users without the need for replacing mouthpieces or the inconvenience of rotation or reorientation after a hand-to-hand exchange.

The methods and apparatus of the present invention are also superior relative to the open-flow breathalyzers. An open-flow breathalyzer is designed such that the breath sample is directly blown onto a sensor or a sensor behind a grill. No means are taken to stabilize the breath flow. Consequently, breath measurements by the open-flow breathalyzers are typically inaccurate and inconsistent. In some embodiments of the present invention, one or more sensors are placed between two breathing entries of a collection tube, either embedded within the tube or inside a device that is in fluid communication with the collection tube. Breath samples taken in at either end of the collection tube travels some distance through the tube and stabilizes inside the device before reaching the one or more sensors. Because of this, the apparatus of the present invention provide accurate measurements of characteristics of a user such as blood alcohol level.

### BRIEF DESCRIPTION OF THE DRAWINGS

FIG. 1A illustrates a closed-flow breathalyzer in accordance with the prior art in which the mouthpiece for breath intake is marked.

FIG. 1B illustrates an open-flow breathalyzer in accordance with the prior art in which an orifice for breath intake is marked.

FIG. 2 is a perspective view of a breathalyzer collection device in accordance with an embodiment of the present invention.

FIG. 3 is a cross-sectional view about line 3-3′ of FIG. 2 in accordance with an embodiment of the present invention.

FIG. 4A is a side cross-sectional longitudinal view about line 4A-4A′ of FIG. 2 in accordance with an embodiment of the present invention.

FIG. 4B is a top cross-sectional view of FIG. 2 about line 4B-4B′ of FIG. 2 in accordance with an embodiment of the present invention.

FIG. 5A is a perspective view of a breathalyzer, in which the casing is not shown, in accordance with an embodiment of the present invention.

FIG. 5B is another perspective view of a breathalyzer, in which the casing is shown, in accordance with an embodiment of the present invention.

Like reference numerals refer to corresponding parts throughout the several views of the drawings.

### DETAILED DESCRIPTION OF THE INVENTION

An apparatus in accordance with the present invention overcomes the disadvantageous features of existing breath analyzing apparatuses by introducing a dual entry collection device for more accurate, compact, convenient and sanitary use. In some embodiments, illumination features are added to the collection device in order to provide visual instruction on where to blow, when to blow into the apparatus, for how long to blow, and to inform the user of the outcome of the breath analysis.

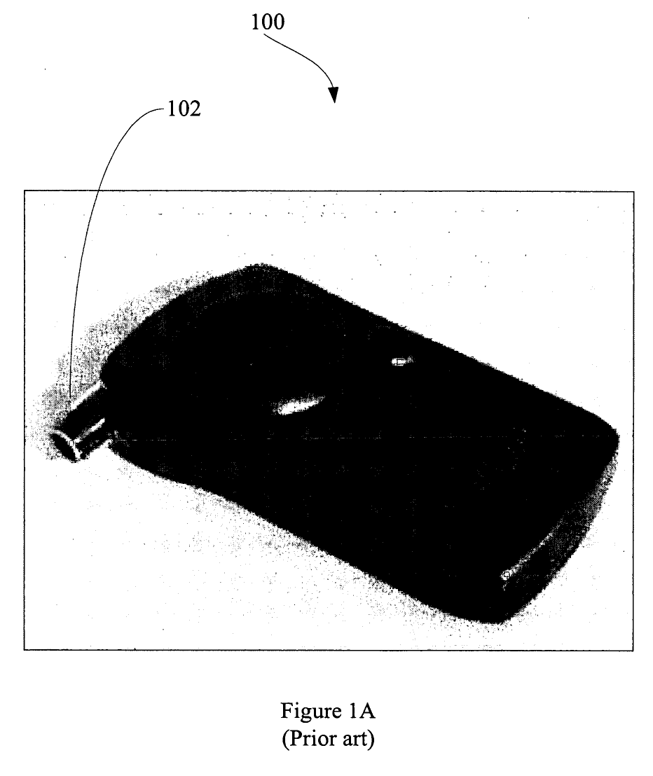

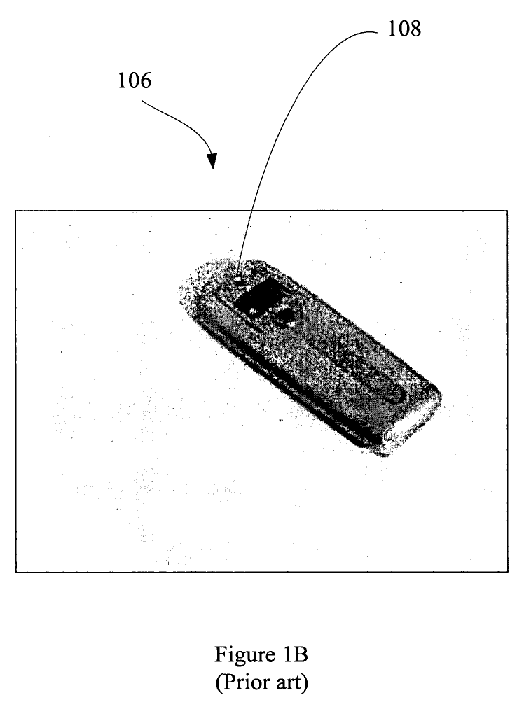

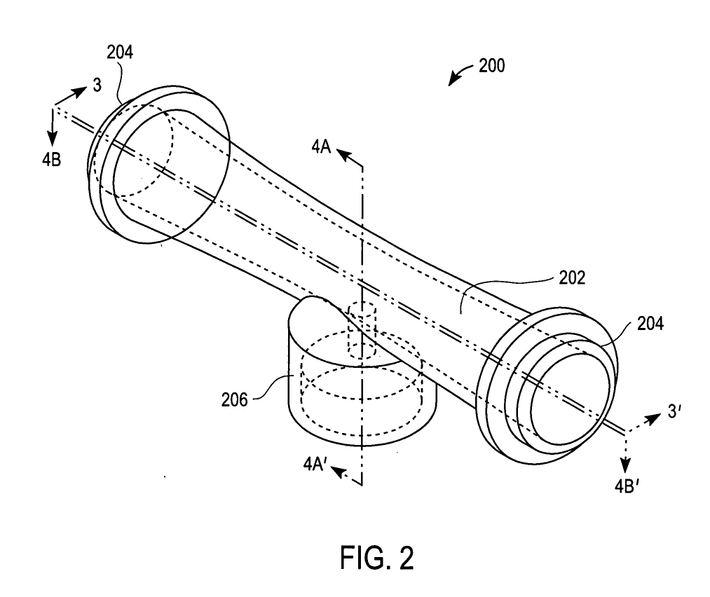

FIG. 2 shows a perspective view of one embodiment of a breathalyzer collection device 200 in accordance with the present invention. The breathalyzer collection device 200 comprises dual entry 204 into a collection tube 202. It will be appreciated that both entries 204 are in fluid communication with each other. When a breath sample is introduced into first entry 204, residual ambient air or excess breath exits the opposing entry. Thus, each entry 204 also serves as an exit. The dual entry architecture of collection device 200 is advantageous over known closed-flow (e.g. FIG. 1A) and open-flow breathalyzers (e.g. FIG. 1B) because it facilitates self-purging of ambient air while at the same time stabilizes airflow over one or more sensors. Although depicted as identical in FIG. 2, there is no requirement that dual entries 204 be identical.

FIG. 2 further illustrates sensor airway 206 of breathalyzer 200. Sensor airway tube 206 is connected to a central region of collection tube 202 between the first and second entry 204. In some embodiments, sensor airway tube 206 houses a sensor device. In some embodiments, sensor airway tube 206 leads to one or more sensors (not shown), thereby providing fluid connection between collection tube 202 and the sensors. Although collection tube 202 is depicted as round, it is not limited to such a shape. In some embodiments, collection tube 202 has a cross-section that is oval, square-like, rectangular-like, or indeed, any possible two-dimensional shape.

In some embodiments, collection tube 202 and airway tube 206 are made of plastic (e.g., a pliable plastic). In some embodiments, collection tube 202 and/or airway tube 206 are made of rubber, a rubberlike material, a rubber derivative, silicone rubber, or an elastomer. In some embodiments, collection tube 202 and/or airway tube 206 are made of natural rubber, vulcanized rubber, a butadiene-styrene polymer such as GR-S, neoprene, nitrile rubbers, butyl, polysulfide rubber, ethylene-propylene rubber, polyurethane rubber, silicone rubber, gutta-percha, and/or balata. In some embodiments collection tube 202 and/or airway tube 206 are made of silicone rubber. Silicone rubber is a rubberlike material having a tensile strength of between 400 lb/in² to 700 lb/in² (2.78 to 4.85×10⁶ N/m²) elongation. In some embodiments, collection tube 202 and airway tube 206 are made of Silastic® silicone rubber (Dow Corning).

In some embodiments, collection tube 202 and airway tube 206 are made out of the same material. In some embodiments, collection tube 202 and airway tube 206 are made out of different materials. As used herein the term elastomer is used to describe both natural and synthetic materials that are elastic or resilient and in general resemble natural rubber in look, feel, and appearance. See, for example, Avallone and Baumeister III, Marks'Standard Handbook for Mechanical Engineers, McGraw Hill, 1987, which is hereby incorporated by reference in its entirety.

In some embodiments, collection tube 202 and airway tube 206 are made out of a plastic or a rubber. In some embodiments, collection tube 202 and/or airway tube 206 are made out of high-density polyethylene, low-density polyethylene, polypropylene, cellulose acetate, vinyl, plasticized vinyl, cellulose acetate butyrate, melamine-formaldehyde, polyester, or nylon. See, for example, Modern Plastics Encyclopedia, McGraw-Hill, which is hereby incorporated by reference in its entirety. In some embodiments, collection tube 202 and/or airway tube 206 are made out of the same or different materials. In some embodiments, at least one of collection tube 202 and airway tube 206 is made of aluminum or an aluminum alloy.

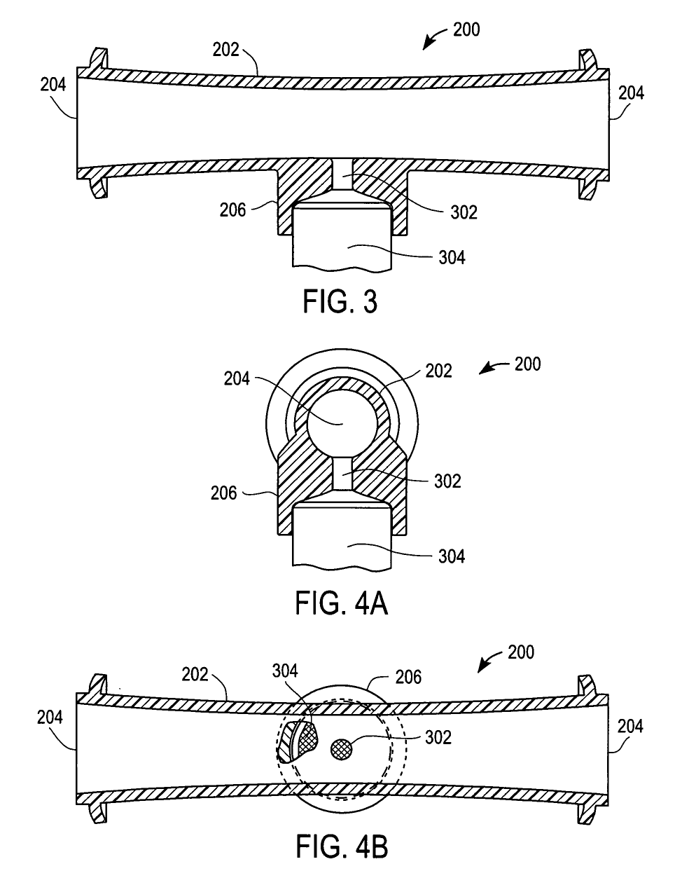

FIG. 3 illustrates a front cross-sectional longitudinal view of a breathalyzer collection device 200 about line 3-3′ of FIG. 2. The figure illustrates how collection tube 202 has a hollow core that fluidly joins entries 204. Thus, when a user blows into one of entries 204, the air sample passes over orifice 302 and is purged out of opposite entry 204. In some embodiments, the diameter of the hollowed core of collection tube 202 decreases toward the center of the tube as depicted in FIG. 3. However, there is no absolute requirement that the hollowed core narrow in this fashion. In some embodiments, the diameter of the hollowed core of collection tube 202 is constant or near constant (subjet to manufacturing imperfections) throughout the length of the collection tube. Collection tube 202 can take any shape so long as dual entries 204 are in fluid communication with each other and so long as collection tube 202 is in fluid communication with sensor airway tube 206.

Referring to FIG. 3, sensor airway tube 206 is in fluid communication with a region of collection tube 202 that is between the first and second breathing entries 204. In some embodiments, sensor airway tube 206 is connected to the center of collection tube 202 as depicted in FIG. 3. However, there is no requirement that sensor airway tube 206 be connected to the center of collection tube 202. Sensor airway tube 206, for example, can be connected to collection tube 202 at a position along collection tube 202 that is closer to one entry 204 than the other entry. In preferred embodiments, however, sensor airway tube 206 is connected to collection tube 202 at or near the center of collection tube 202 so that a breath sample from either breathing entry 204 gets stabilized in the same manner prior to reaching a sensor that is located inside or near sensor airway tube 206. Furthermore, positioning sensor airway tube 206 so that it is at or near the center of collection tube 202 guarantees that the same breath analysis result will be achieved regardless of which entry 204 is used by a user to provide a breath sample.

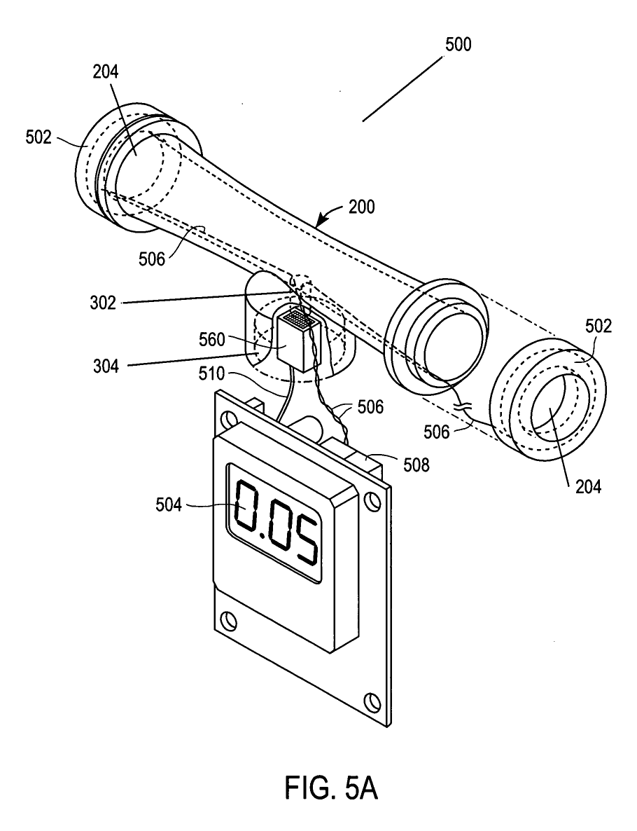

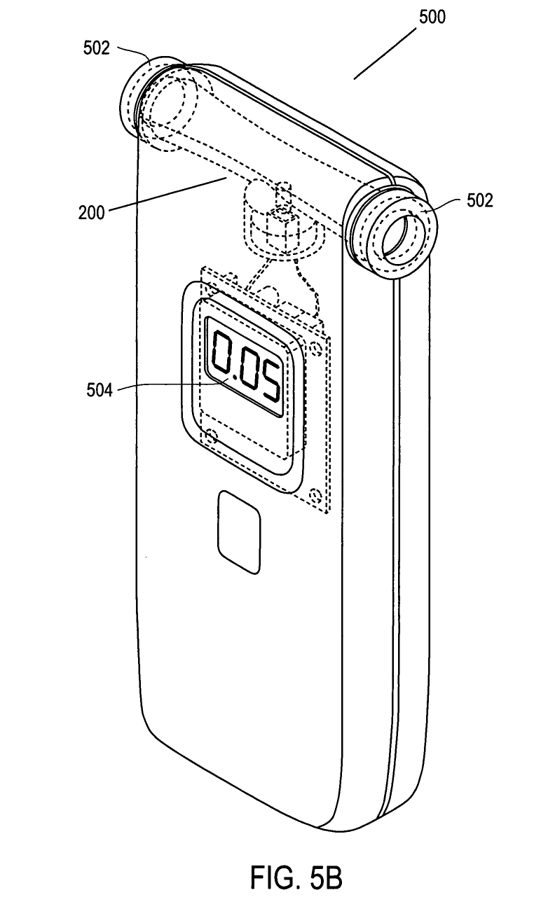

In some embodiments, sensor airway tube 206 comprises a first portion 302 having a first inner diameter and a second portion 304 having a second inner diameter, such that the first portion of the sensor airway tube 206 is attached to collection tube 202. In some embodiments, the first inner diameter is smaller than the second inner diameter. Such an architecture is advantageous because the first inner diameter (the inner diameter of portion 302 of sensor airway tube 206) constricts the amount of air that passes from collection tube 202 into sensor airway tube 206. In some embodiments, the inner diameter or first portion 302 is the size of a pinhole. In preferred embodiments, the inner diameter of second portion 304 is considerably larger than the size of the pinhole. In fact, in some embodiments, second portion 304 is large enough to house a sensor 560 (FIG. 5A). Sensor 560 is capable of detecting and quantifying some measurable aspect of a breath sample such as alcohol content. Alternatively, sensor 560 is placed inside collection tube 202 approximately in the center, or proximate but outside of sensor airway tube 206. In some embodiments, there are multiple sensors 560.

An example of sensor 560 includes the SB-30 gas sensor for alcohol detection that is commercially available from FIS Inc. Such a sensor is a tin-dioxide semiconductor gas sensor. Another example of a sensor 560 is the SB-AQ4 sensor commercially available from FIS Inc. that detects cigarette smoke. Still another example of a sensor 560 is a sensor that tests for gases associated with bad breath. A full product list of FIS Inc. sensors, any of which can be used as a sensor 560 in the present invention, is available at http://www.fisinc.cojp/04_prod.htm.

In some embodiments in accordance with the present invention, sensor 560 detects characteristics other than alcohol content. For instance, in some embodiments, sensor 560 can detect volatile sulfur compounds indicative of bad breath. Such volatile sulfur compounds include hydrogen sulfide, methyl mercaptan, and dimethyl sulfide. Bad breath, also known as halitosis or mouth malador, refers to the unpleasant smell from a person's mouth. Notwithstanding adverse social implications, bad breath can have serious medical causes including foul-smelling bacterial infections, chronic mouth inflammation, poor digestion, dental cavities, and mouth, tongue, or gum infections. In addition, other serious illnesses, such as bronchiectasis, liver or kidney failure, and diabetic coma, can all give mouth malodors. Analyzing breath samples using the present invention provides a mechanism, therefore, for monitoring personal health.

Research has indicated that hydrogen sulfide, methyl mercaptan, other thiols, and dimethyl sulfide, collectively referred to as volatile sulfur compounds (VSC), are the principal malodorants in chronic halitosis. See, Pratten et al., 2003, Arch Oral Biol 48, 737; Seemann et al., 2001, J Clin Dent 12:104-107; Frascella et al., 2000, Compend Contin Educ Dent 21, 241-244; Neiders 30, 295-301; Ben-Aryeh et al., 1998, Am J Otolaryngol 19, 8-11; Delanghe et al., 1997, Acta Otorhinolaryngol Belg 51, 43-48; Richter et al., 1996, Compend Contin Educ Dent 17, 370-386; and Touyz, 1993, J Can Dent Assoc 59:607-610, each of which is hereby incorporated by reference in its entirety. Accordingly, in some embodiments, sensor 560 is an electrochemical voltammetric sensor. Such a sensor is, for example, described in U.S. Pat. No. 4,017,373, which is hereby incorporated by reference in it entirety. In some embodiments, sensor 560 is an electrochemical voltammetric sensor having an accuracy of 5 ppb, and a lag time of less than 1 second. Such a sensor has been incorporated into the Halimeter® from Interscan Corporation (Chatsworth, Calif.). Sensors of similar mechanism may be incorporated into embodiments of the present invention for detecting bad breath.

Referring to FIG. 3, the constrained inner diameter of first portion 302 of sensor airway tube is referred to as a “pin-hole” airway. It is the region of sensor airway tube 206 that provides fluid communication between collection tube 202 and the one or more sensors 560 in or proximate to sensor airway tube 206. After a user blows into collection tube 202 through an entry 204, a very small portion of the resulting breath sample goes through the pin-hole airway. This is because the diameter of the pin-hole airway, the inner diameter of first portion 302, is much smaller than the diameter of the inner core of collection tube 202. Thus, most of the breath sample is simply blown out the other entry 204. Although just a small portion of the breath sample enters the pin-hole airway into sensor airway tube 206, the architecture is advantageous because this small sample is highly stabilized and thus is thoroughly representative of the breath composition of the user. The air sample is further stabilized inside the “pin-hole” 302 before being analyzed by the one or more sensors 560.

FIG. 4A is a side cross-sectional view about line 4A-4A′ of FIG. 2. FIG. 4A depicts sensor airway tube 206 from an angle that is ninety degrees with respect to FIG. 3. The figure illustrates how the pin-hole inner diameter of the first portion 302 of sensor airway tube 206 provides fluid communication between collection tube 202 and a sensor that is either inside or proximate to sensor airway tube 206. FIG. 4B is a top cross-sectional view about line 4B-4B′ of FIG. 2, showing the pin-hole inner diameter of the first portion 302 as well as the inner diameter of the second portion 304 of sensor airway tube 206. Although the pin-hole inner diameter of first portion 302 of sensor airway tube 206 is depicted as a round tube-like structure, there is no absolute requirement that such a configuration be adopted. In fact, the inner core of the first portion of sensor airway tube can have any shape that provides fluid communication between collection tube 202 and sensor airway tube 206.

FIG. 5A provides more details of additional elements of a breathalyzer 500 in accordance with embodiments of the present invention. In the embodiment illustrated in FIG. 5A, breathalyzer 500 comprises the breathalyzer collection device 200, a sensor device 560, a display device 504, and illumination device 502. More specifically, entries 204 are equipped with lights 502. Wiring 506 feeds back to a control unit 508. Control unit 508 controls the illumination of lights 502. In some embodiments, each light 502 has a ring-like shape. In some embodiments in accordance with the present invention, such illumination rings are attached to breathing entries 204 of collection tube 202. The breathalyzer further includes a sensor 560 that is housed in the inner diameter of the second portion 304 of sensor airway tube 206 through connection 510. When a breath sample reaches sensor 560, the sensor produces a signal that is displayed on display 504. In some embodiments, display 504 is an LCD display. In some embodiments, display 504 is any kind of display that can display alphanumeric characters.

In some embodiments, control unit 508 is one or more application specific integrated circuits (ASICs) and/or field-programmable gate arrays (FPGAs). In some embodiments, control unit 508 is implemented as one or more digital signal processors (DSPs). In such embodiments, control unit 508 is considered any combination of chips, including any combination of ASICs, FPGAs, DSPs, or other forms of microchips known in the art. In general, any type of microarchitecture that can control lights 502, display 504, and sensor 560 is a suitable control unit 508.

In some embodiments, lights 502 are simply a transparent plastic and illumination is fed to the lights by fiberoptic channels (not shown) from a series of LEDS (not shown). In such embodiments, control unit 508 controls which LEDs are illuminated. For example, in some embodiments, a bank of LEDs is housed within the casing of breathalyzer 500. The bank includes different colored LEDs. For example, in some embodiments, the bank of LEDs includes red LEDs and green LEDs. In some embodiments, the bank of LEDs includes red, green, and blue LEDs. In yet other embodiments, the bank of LEDs includes one or more LEDs that can change color. Thus, control unit 508 is capable of illuminating lights 502 with different colored lights. Such an architecture is highly advantageous because it can be used to encode instructions and communicate information to a user. For example, consider the case in which there is a bank of LEDs that includes red, green, and blue LEDs. Control unit 508 can illuminate the blue LEDs thereby causing lights 502 to shine blue. This is a signal to the user that it is now permissible to start blowing into an entry 204 of collection tube 202. The breath sample is analyzed by sensor 560 and the result communicated to control unit 508. Control unit 508 determines whether a physical feature of the breath sample exceeds a threshold value. If it does, than control unit 508 powers on the red LEDs and powers off the blue LEDs. If the physical feature of the breath sample does not exceed a threshold value, than control unit 508 powers on the green LEDs and powers off the blue LEDs. Representative LEDs that can be used in the present invention are disclosed in the section below entitled exemplary LEDs. In some embodiments, this physical feature is alcohol content in the breath sample.

In some embodiments, there is a power/start/reset button (not shown) that is in electrical communication with control unit 508. The power/start/reset button can be used by a user when the user desires to have a sample read. In an exemplary embodiment that includes a reset button, the user presses the power/start/reset button and waits for lights 502 to shine blue indicating that it is clear to start blowing. The user continues to blow until control unit 508 powers off the lights 502. The user then waits to see whether the lights 502 shine green, indicating that all is clear (the physical feature is below a predetermined threshold value) or to shine red (a warning that the physical feature is above a predetermined value. Those of skill in the art will appreciate that any number of variations of the above scenario are possible. For example, the bank of LEDs can include multiple colors with each color representing one step along a gradient between a very low measurement for a physical feature to a very high value for the physical feature. For example, shades of green can represent varying low values for a measured physical feature, varying shades of yellow can represent varying intermediate values for a measured physical feature, and varying shades of red can represent varying high values for a measured physical feature in a breath sample. In such an embodiments, control unit 506 can use, for example, a look-up table that equates specific LED colors with specific ranges for the measured physical feature.

It will be further appreciated that lights 502 do not require LEDs in order to communicate instructions and results to a user independent of display 504. While display 504 can display instructions to the user on when to blow, and a digital representation of a measured value of a physical feature in a breath sample, lights 502 can also communicate such information even if only a single color light is used. For example, lights 502 can flash a constant light after the user has pressed a reset button. Then, to convey the results of a measured physical feature in the breath sample, lights 502 can be made to flash when the measured physical feature exceeds a threshold value or to turn off altogether when the measured physical feature does not exceed a threshold value.

In one use case scenario in accordance with some embodiments of the present invention, a user accessible start button is located near display 504. A message, such as “please press the start button” can be displayed on display 504 prior to measurement. After a user pushes the start button, control unit 508 causes lights 502 to flash blue light. Meanwhile, display 504 reads “please breathe into the collection tube.” A user then breathes into either entry 204 of collection tube 202. If an inadequate breathe sample is provided, a warning message is displayed on display 504. As an additional measure, control unit 508 can cause lights 502 to stop flashing blue and turn yellow. The user can also be reminded to clear the system, for example, by pushing the start button again, to reinitiate the measuring process. If an adequate breath sample is collected, sensor 560 measures a physical feature of the breath sample (e.g., alcohol content) and signals control device 508 that such a sample has been acquired. The value of the measured physical feature is displayed on display device 504. Alternatively, in some embodiments in accordance with the present invention, instructions on display 504 can be icons, texts, or a combination of icons or texts, or even animated icons or video clips. Advantageously, in some embodiments in accordance with the present invention, lights 502 are also used to communicate this result (e.g., by shining either a green light for normal values of the physical attribute or a red light for values of the physical attribute that are outside of threshold limits).

FIG. 5B illustrates a breathalyzer 500 complete with a casing. As depicted, breathalyzer 500 includes collection device 200, display 504, and illumination device 502 in a casing. The casing can be made of plastic, aluminum, steel, or any other material used to encase portable electronics that is known in the art. Furthermore, the casing can be made out of any the materials used to make collection tube 202 and/or airway tube 206.

An exemplary sequence for conducting an alcohol breath analysis using an embodiment of breathalyzer 500 in accordance with the present invention will now be described. First, a user presses a power/start/reset button to turn on the system. Lights 502 are illuminated and a text message and or icons are displayed on display 504 to give user directions. The control unit 508 counts down approximately five to ten seconds by displaying appropriate messages on display 504. Lights 502 flash. During this time, sensor 560 is primed. In some embodiments in accordance with the present invention, the lag time can be shorter than five seconds, between five and ten seconds, or longer than ten seconds. When the system is ready for a breath sample, control unit 508 causes illumination device 502 to emit a visible signal by either changing its color or by flashing. The user breathes into either entry 204 of collection tube 202. After control unit 508 determines that the user has provided an adequate breath sample, the unit 508 causes lights 502 to illuminate differently thereby instructing the user to stop blowing into breathalyzer 200. The user's blood alcohol level is then calculated and displayed on display 504. When control unit 508 determines that an inadequate breath sample has been provides, unit 508 causes lights 502 to emit a visible signal (e.g. by the use of a different color or flashing sequence), and display 504 provides a suitable error message to remind the user to provide another sample. After measurements are complete, the user can press and hold down the power button to turn off the system. In some embodiments in accordance with the present invention, the breathalyzer unit 500 can shut off automatically after a period of inactivity or idleness. In some embodiments, a speaker is used to emit messages in conjunction with light 502. For example, one form of alarm can sound when the breathalyzer 500 is ready to accept a sample, another form of alarm can sound when the breath sample is inadequate, and still another form of alarm can sound when the a physical attribute in the measure sample exceeds a threshold value.

In some embodiments, breathalyzer 500 is integrated into handheld devices, such as a cellular telephone (cell phone) or a personal digital assistant (PDA). In addition, these handheld devices may be in global positioning systems (GPS), walkie-talkies, radios, music players, MP3 devices, car key holders, police batons, flashlights, karaoke microphones, iPods, drink containers, watches, cigarette lighters, dictaphones, voice recorders, traffic sticks, video players, portable multimedia devices, tip trays, pagers, or coasters. Breathalyzer 500 can further be incorporated into a variety of consumer, law enforcement, healthcare, and commercial products and applications.

The length of a collection tube 202 directly correlates with its ability to stabilize breath samples. The versatile nature of the designs in accordance with the present invention allows a collection tube 202 to utilize the inherent lengths, heights, depths, or widths of handheld devices to add more accuracy inconspicuously, without compromising the compactness of the original devices. Also advantageously, the embodiments in accordance with the present invention do not require disposable mouthpieces, sterilization, or cleaning after each use. They are sanitary, cost effective, convenient, and environmentally responsible.

For the purpose of illustration, breathalyzers are used to convey the concepts of the present invention. The designs embodied in the present invention are by no means limited to breath alcohol level testing. The scope of this invention covers any breath collection and analysis devices including devices for testing bad breath or metabolic gas exchange.

### Exemplary Leds

Exemplary LEDs that can be used to power lights 502 include but are not limited to the LEDs described in this section. One example of an LED is yellow is gallium arsenide phosphide (GaAsP) with a low arsenic/phosphorus ratio of about 20/80 to 10/90, on a gallium phosphide substrate. The color is usually orangish yellow (“amber”) with a dominant wavelength around 590 nm or in the upper 580's. Typical drive current is 20 mA and maximum drive current is usually 30 mA. Typical voltage drop at 20 mA is about 2 volts. Efficiency is usually maximized at currents near or over 20 mA. Still another LED is high efficiency yellow GaAlAsP, with overall luminous efficacy around roughly 0.5 lumen per watt.

Yet another suitable LED is green LEDs based upon gallium phosphide, in some instances nitrogen-doped for maximum efficiency along with a very yellowish shade of green (dominant wavelength usually 565-570 nm). Efficiency is usually maximized at currents near or over 20 mA, but a few more efficient ones have efficiency peaking around 15 mA at about half a lumen per watt are available. Typical voltage drop at 20 mA is about 2.1 volts. Maximum drive current is usually 30 mA.

Still another LED is high efficiency green LEDs based upon gallium aluminum phosphide. Such LEDs have characteristics similar to those of the nitrogen-doped gallium phosphide ones, except the overall luminous efficacy is higher—around 1 lumen/watt. Still another LED is “pure green” LEDs gallium phosphide. Such LEDs are yellowish green, but less yellow than usual with dominant wavelength in the 550's of nm. Hewlett Packard refers to this color as “emerald green.” Efficiency is maximized at currents near or over 20 mA. Maximum drive current is usually 30 mA.

Still another suitable LED is indium gallium nitride (InGaN) ultrabright blue and green LEDs. Most of these are a slightly turquoisish blue (dominant wavelength around 470 nm), a slightly whitish green (dominant wavelength around 525-527 nm), or “traffic signal” bluish green. These are also made in deeper shades of blue, blue-violet and UV.

Still another suitable LED are white LEDs, which are in fact blue LED chips covered with a phosphor that absorbs some of the blue light and fluoresces with a broad spectral output ranging from mid-green to mid-red. Lumileds is now producing units achieving 25 lumens/watt and ones based on Green blue chips may soon achieve 25-30 lumens/watt. The spectrum of these white LEDs consists of the LED band in the mid-blue plus the phosphor band from mid-green to mid-red. The spectrum runs low in far red and blue-green, high in mid-blue, low in violet-blue and really low in the violet.

Yet another LED suitable for uses in the present invention is wideband GaN Blue. This is an ultrabright blue LED, pioneered by Nichia in the mid 1990's. The color is a slightly whitish blue. The spectrum has significant content through the violet, blue, and green regions. The peak wavelength is typically 450 nm. Maximum drive current is optimistically 30 mA. These LEDs are unusually intolerant of voltages and currents in excess of their ratings and are considered static sensitive.

Still another LED that can be used in the present invention is silicon carbide blue. These are the original commercially successful blue LEDs pioneered by Cree in the early 1990's. The color is turquoise blue at 10-20 mA and more aqua at lower currents. The spectrum is broad, like that of wideband 450 nm GaN blue LEDs but with less violet. Maximum drive current is optimistically 50 mA and usually quite safely 30 mA. The maximum chip temperature of silicon carbide old type blue LEDs is 150 degrees C., higher than that of other LEDs.

Still another LED that can be used in the present invention is violetish Blue GaN on a SiC substrate (Cree “standard blue”). Even though the overall luminous efficacy is only around half a lumen per watt, the intense slightly violetish, slightly whitish blue color is useful. The spectrum is broadband but is strong in the mid-blue to violet, peaking around 428-430 nm. According to Cree, the dominant wavelength is 466 nm. The efficiency does not vary much with current from a few mA to 20 mA, but higher currents extremely slightly favor a deeper blue color. Maximum drive current is optimistically 30 mA. Typical drive current should be 10 mA. The voltage drop at 20 mA is anywhere from 3.8 to about 5 volts, and generally higher in earlier models.

In general LEDs produced by manufacturers that included, but are not limited to Siemens, Nichia, Agilent, Lumileds, and Cree can be used in the present invention.

### Conclusion

Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

What is claimed is:

1. A breath analyzing method utilizing an apparatus comprising a collection device, wherein
(i) the collection device has at least a first breathing entry and a second breathing entry;
(ii) the first breathing entry and the second breathing entry are in fluid communication with each other; and
(iii) the collection device has a center between the first breathing entry and the second breathing entry, the method comprising:
breathing into either said first breathing entry or said second breathing entry thereby providing a breath sample of a user;
releasing exhausted breath from either said second breathing entry or said first breathing entry thereby purging the collection device; and
obtaining a reading from said apparatus that is indicative of a characteristic of the user.

2. The method of claim 1, wherein the apparatus further comprises one or more sensors collectively configured to measure a physical feature of the breath sample, wherein the sensors are located between the first breathing entry and the second breathing entry of the collection device, and wherein the method further comprises:
measuring, between the first breathing entry and the second breathing entry of the collection device, a physical feature of the breath sample.

3. The method of claim 2, wherein the apparatus further comprises an airway tube in fluid communication with the collection device and wherein the one or more sensors are located in the airway tube.

4. The method of claim 2, wherein the physical feature is an alcohol content of the breath sample.

5. The method of claim 1, wherein the characteristic is a blood alcohol level of the user.

6. The method of claim 1, wherein the characteristic is a metabolic gas content of the user.

7. The method of claim 1, wherein the characteristic is bad breath.

8. The method of claim 1, wherein the collection device is a tube and the first breathing entry is a first opening of the tube and the second breathing entry is a second opening of the tube.

9. The method of claim 1, wherein the breathing is in response to a first visible signal.

10. The method of claim 9, wherein said first visible signal is a first type of illumination of at least one of the breathing entries.

11. The method of claim 10, wherein said first type of illumination is a blue light.

12. The method of claim 1, wherein the obtaining is communicated by a second visible signal.

13. The method of claim 12, wherein said second visible signal is a second type of illumination of the first breathing entry and the second breathing entry.

14. The method of claim 13, wherein said second type of illumination is a red light or a green light.

15. A breath analyzing apparatus comprising a collection device, wherein the collection device comprises:
(i) a first breathing entry;
(ii) a second breathing entry, wherein the first breathing entry and the second breathing entry are in fluid communication with each other, and wherein the collection device has a center between the first breathing entry and the second breathing entry; and
(iii) one or more sensors collectively configured to measure a physical feature of a breath sample, wherein the one or more sensors are in fluid communication with a region that is between the first breathing entry and the second breathing entry of the collection device.

16. The breath analyzing apparatus of claim 15, wherein the collection device is a tube and the first breathing entry is a first opening of the tube and the second breathing entry is a second opening of the tube.

17. The breath analyzing apparatus of claim 15, the apparatus further comprising a sensor airway tube that is in fluid communication with the collection device, and wherein the sensor airway tube houses said one or more sensors.

18. The breath analyzing apparatus of claim 16, wherein the sensor airway tube comprises:
a first portion having a first inner diameter; and
a second portion having a second inner diameter, and wherein
the first portion of said sensor airway tube is attached to said collection device.

19. The breath analyzing apparatus of claim 18, wherein the first inner diameter is smaller than the second inner diameter.

20. The breath analyzing apparatus of claim 15, the collection device further comprising:
a first illumination source connected to the first breathing entry;
a second illumination source connected to the second breathing entry; and
a control mechanism in electrical communication with said first illumination source and said second illumination source, and wherein the control mechanism is configured to:
cause said first illumination source and said second illumination source to emit a first visible signal indicating a time for a user to commence breathing into said first breathing entry or said second breathing entry; and
cause said first illumination source and said second illumination source to emit a second visible signal indicating a characteristic of said user.

21. The breath analyzing apparatus of claim of 20, wherein
the first visible signal is a blue light; and
the second visible signal is a green light or a red light.

22. The breath analyzing apparatus of claim of 20, wherein the control mechanism is configured to continue emitting said first visible signal during a breath measurement.

23. The breath analyzing apparatus of claim of 20, wherein the control mechanism is configured to emit a signal indicating insufficient breath input.

24. The breath analyzing apparatus of claim of 22, wherein the signal is an audible alarm.

25. The breath analyzing apparatus of claim of 20, wherein the apparatus further comprises a casing that houses said collection device and wherein a display is mounted on said casing, the display in electrical communication with the control mechanism, and wherein said signal is a message on said display.

26. The breath analyzing apparatus of claim 15, wherein said apparatus is ambidextrous.

27. The breath analyzing apparatus of claim 15, wherein said apparatus is a handheld device.

28. The breath analyzing apparatus of claim 15, wherein the apparatus is a cell phone or a personal digital assistant (PDA).

29. The breath analyzing apparatus of claim 15, wherein the apparatus is a global positioning systems (GPS), a walkie-talkie, a radio, a music player, an MP3 device, a car key holder, a police baton, a flashlight, a karaoke microphone, an iPod, a drink container, a watch, a cigarette lighter, a dictaphone, a voice recorder, a traffic stick, a video player, a portable multimedia device, a tip tray, a pager, or a coaster.

30. The breath analyzing apparatus of claim 15, wherein the apparatus is a handheld device having a casing, and wherein the collection device is housed by said casing such that the first breathing entry forms a first entry in said casing and said second breathing entry forms a second entry in said casing.
